(19) United States
(12) Patent Application Publication
(10) Pub. No.: US 2024/0091028 A1
(43) Pub. Date: Mar. 21, 2024

(54) SYSTEM AND METHOD FOR GUIDING INTERBODY SPACER BETWEEN VERTEBRAL BODIES

(71) Applicant: LIGA ACE CORPORATION, Shizuoka City (JP)

(72) Inventor: Yoshio Shinozaki, Shizuoka (JP)

(21) Appl. No.: 18/520,868

(22) Filed: Nov. 28, 2023

Related U.S. Application Data
(63) Continuation of application No. 17/610,834, filed on Nov. 12, 2021.

(30) Foreign Application Priority Data
Aug. 30, 2019 (JP) 2019-157595

(51) Int. Cl. A61F 2/46; A61F 2/44

(52) U.S. Cl. CPC A61F 2/4611; A61F 2/4465; A61F 2002/30387

(57) ABSTRACT

A system for treating a spinal disease by placement of an interbody spacer between vertebrae comprises a horizontally curved interbody spacer and a guiding tool. The interbody spacer includes a pair of contact surfaces for contact with the vertebrae, a convex ventral surface that connects the contact surfaces, and a concave dorsal surface that connects the contact surfaces. The interbody spacer includes an engagement portion, located along the direction in which the interbody spacer is curved, in the ventral surface and/or the dorsal surface. The guiding tool for guiding the interbody spacer to a predetermined position between the vertebrae includes, on a distal end, a guide rail portion to be fitted into one of the engagement portions. The radius of curvature of the guide rail portion in plan view is substantially the same as the radius of curvature of the ventral surface or the dorsal surface of the interbody spacer.

### CROSS-REFERENCE TO RELATED APPLICATION

This application is a continuation of U.S. application Ser. No. 17/610,834 filed Nov. 12, 2021, and claims priority based on Japanese Patent Application No. 2019-157595 filed on Aug. 30, 2019. The entire contents of Japanese Patent Application No. 2019-157595 are incorporated herein by reference.

### FIELD OF THE INVENTION

The present invention pertains to a system and method for guiding an interbody spacer between vertebral bodies.

### BACKGROUND OF THE INVENTION

It is essential to use an interbody spacer for obtaining bony fusion by means of PLIF (Posterior Lumbar Interbody Fusion), and it is possible to further stabilize the intervertebral space and achieve firmer fusion by inserting a spacer which is as large as possible and conforms to the shape of an intervertebral space. In addition, a larger spacer is expected to be capable of being filled with a greater quantity of graft bone, etc., and is considered to contribute to future intervertebral bony fusion.

There is concern that the insertion of an interbody spacer only from a unilateral side by using a conventional box-type interbody spacer by means of a unilateral approach and unilateral facetectomy may cause contralateral intervertebral instability to remain, which may be detrimental to bony fusion. There is also concern that the insertion of a box-type interbody spacer from bilateral sides by means of a bilateral approach and bilateral facetectomy may further induce intervertebral instability. In contrast, the insertion of an interbody spacer or the insertion of a conventional boomerang-shaped interbody spacer to the contralateral side by means of a unilateral approach and unilateral facetectomy is a reasonable method as it is possible to provide support as far as the contralateral side while preserving contralateral intervertebral joints. Interbody spacers are described in Japanese Patents JP 2012-532693A, JP 2016-512107A, and

### JP 2019-517891A

### SUMMARY OF THE INVENTION

The problem to be solved by the invention is as follows. When inserting and placing an interbody spacer between vertebral bodies, fenestration is performed in the annulus fibrosus of an intervertebral disc 2 between vertebral bodies 1 shown in FIG. 1, and the nucleus pulposus therein is removed, and then the interbody spacer is inserted and placed in the intervertebral space after the removal. As the spinal nervous system and blood vessels run near the intervertebral disc 2, extreme care is required when carrying out the surgical operation.

As the spinal column performs the function of supporting body weight, an interbody spacer is required to have high rigidity and to cover a large area between vertebral bodies, and furthermore, as the surgical field is narrow and there are the nervous system and blood vessels running nearby, skills and surgical systems are required for placing the interbody spacer in an appropriate position while avoiding the nervous system and blood vessels.

As mentioned above, the insertion of a conventional boomerang-shaped interbody spacer is a reasonable method because it is possible to provide support as far as the contralateral side while preserving contralateral intervertebral joints. However, use of an intraoperative fluoroscopic device and highly advanced techniques are required when sending the interbody spacer to the contralateral side.

In addition, regarding a conventional interbody spacer, the size of the interbody spacer that can be inserted through a narrow insertion opening is limited. For this reason, interbody spacers having flexibility and insertion tools and the like that can be oriented within the intervertebral space have been developed to date, but many of those systems are structurally complicated and thereby have vulnerability, and thus few systems have been put into practical use.

In the system described in Japanese Patent JP 2012532693A, interbody spacers are connected to each other by a flexible bridge. The mechanism is configured such that the interbody spacers are inserted into an inserter tube, allowed to pass through the inserter tube to be inserted and placed between the vertebral bodies. The structures of the interbody spacers are complicated, which reduce the rigidity of the interbody spacers. Further, in the disclosed embodiments, the insertion tool includes a guide rail for guiding the bottom surface of each interbody spacer and a guide rail for guiding the bottom surface, superior surface, and side surface of each interbody spacer, and thus there remains a problem to be solved in guiding the interbody spacer to a narrow surgical field.

In the system described in Japanese Patent JP 2016512107A, an interbody spacer has an expandable function. The system is configured such that after placing the interbody spacer between vertebral bodies in a non-expanded state by using a tool, the interbody spacer is expanded within the intervertebral space. Numerous parts are connected by pins, etc. in the interbody spacer, so that the structure is complicated, and the rigidity is reduced. In addition, it is required that the interbody spacer be placed in a non-expanded state between the vertebral bodies and then expanded, and thus the system is complicated, and a highly advanced skill of a surgeon is required in surgical operation.

In the system described in Japanese Patent JP 2019517891A, an interbody spacer is one integrated piece and has rigidity. A tool for placing the interbody spacer includes a gripping portion for gripping a pin that is provided in the interbody spacer, and a surgeon is required to place the interbody spacer in a narrow surgical field while adjusting the orientation thereof, and accordingly there is a problem that the surgical operation is difficult.

It is an object of the present invention to provide a simple system capable of easily placing an interbody spacer accurately in a predetermined position between vertebral bodies even in a narrow surgical field.

The means for solving the above-described problem is as follows. The present invention is a system for treating a spinal disease. The system comprises an interbody spacer which is curved in a horizontal direction and used by being inserted between vertebral bodies. The interbody spacer includes a pair of contact surfaces to be in contact with each of the vertebral bodies, and includes a ventral surface which is a side surface on a ventral side, having a convex shape in plan view, and which connects the pair of contact surfaces on the ventral side, and a dorsal surface which is a side surface on a dorsal side having a concave shape in plan view and which connects the pair of contact surfaces on the dorsal side. The interbody spacer includes an engagement portion, which is located along the direction in which the interbody spacer is curved, in the ventral surface and/or the dorsal surface. A guiding tool for guiding the interbody spacer to a predetermined position between the vertebral bodies. The guiding tool includes, on the distal end side, a guide rail portion to be fitted into either one of the engagement portion in the ventral surface and the engagement portion in the dorsal surface. The guide rail portion has a radius of curvature in plan view which is substantially the same as the radius of curvature of the ventral surface or the dorsal surface in plan view. Either one of the transverse cross section of the engagement portion and the transverse cross section of the guide rail portion has a concave shape, while the other has a convex shape that corresponds to the concave shape. The concave shape includes a portion having a maximum width that is greater than the opening width of the concave shape that is opposed to the convex shape.

In the system of the present invention, the engagement portion is provided along the direction in which the interbody spacer is curved in the ventral surface and/or the dorsal surface of the interbody spacer. The guiding tool includes, on the distal end side, the guide rail portion to be fitted into either one of the engagement portion in the ventral surface and the engagement portion in the dorsal surface. The radius of curvature of the guide rail portion in plan view is substantially the same as the radius of curvature of the ventral surface or the dorsal surface in plan view, so that the interbody spacer can be moved along the guide rail portion. Once the guiding tool is placed in a predetermined position, it is possible to guide the interbody spacer accurately and easily to a predetermined position between vertebral bodies.

By using the present system, for example, when the insertion is performed by a TLIF (Transforaminal Lumbar Interbody Fusion) approach, rotation is made approximately around the dura mater, and insertion is made from the lateral edge of the intervertebral space to be in contact with the anterior edge and the lateral edge on the contralateral side. By inserting the interbody spacer along the guide rail portion of the guiding tool that is placed in advance between the vertebral bodies, it is possible to insert the interbody spacer safely to the optimal position on the contralateral side more accurately and easily.

In an embodiment of the present invention described above, the guide rail portion has a transverse width equal to or smaller than the height of the ventral surface and/or the dorsal surface of the interbody spacer. By setting the transverse width dimension of the guide rail portion to be equal to or smaller than the height of the ventral surface and/or the dorsal surface of the interbody spacer, it is possible to place the interbody spacer smoothly in a predetermined position between vertebral bodies even in a narrow surgical field.

In either of the above-described embodiments of the present invention, the transverse cross-section of the engagement portion can be a T-shaped concave cross-section, and the transverse cross-section of the guide rail portion can be a T-shaped convex cross-section.

When the transverse cross-section of the engagement portion on the interbody spacer side is a T-shaped concave cross-section, while the transverse cross-section of the guide rail portion is a T-shaped convex cross-section, the interbody spacer can be guided along the convex-shaped rail of the guide rail portion so as to be easily placed in a predetermined position between the vertebral bodies.

In either of the first two embodiments of the present invention the transverse cross-section of the engagement portion can be a T-shaped convex cross-section, and the transverse cross-section of the guide rail portion can be a T-shaped concave cross-section.

Alternatively, if the transverse cross-section of the engagement portion on the interbody spacer side is a T-shaped convex cross-section, while the transverse cross section of the guide rail portion is a T-shaped concave cross section, the interbody spacer can be guided along the concave shaped rail of the guide rail portion so as to be easily placed in a predetermined position between the vertebral bodies.

Another aspect of the present invention is the interbody spacer of any of the above-described systems. By using such an interbody spacer, it is possible to place the interbody spacer accurately and easily in a predetermined position between vertebral bodies.

Still another aspect of the present invention is the guiding tool of any of the above-described systems. By using such a guiding tool, it is possible to place the interbody spacer accurately and easily in a predetermined position between vertebral bodies.

The system of the present invention has a simple configuration, and thus it is possible to place the interbody spacer accurately and easily in a predetermined position between vertebral bodies even in a narrow surgical field.

### BRIEF DESCRIPTION OF THE DRAWINGS

FIG. 1 is a side view of the spine (the lumbar spine);

FIG. 2 is a perspective view in which a guiding tool is placed between vertebral bodies;

FIG. 3 is a perspective view of an interbody spacer according to the present invention;

FIG. 4 is a plan view, a medial side view, a lateral side view, and a ventral side view of the interbody spacer;

FIG. 5 is a perspective view of an example of a guiding tool according to the present invention;

FIG. 6 is a plan view of the guiding tool of FIG. 5;

FIG. 7 is a perspective view of another example of a guiding tool according to the present invention;

FIG. 8 is a plan view of the guiding tool of FIG. 7,

FIG. 9 is a schematic view showing the manner in which two interbody spacers according to the present invention are symmetrically placed between vertebral bodies, by engaging the inner periphery of the guide rail portion of the guiding tool of FIGS. 5 and 6 with the ventral surface of each of the interbody spacers.;

FIG. 10 is a schematic view showing the manner in which two interbody spacers according to the present invention are symmetrically placed between vertebral bodies, by engaging the outer periphery of the guide rail portion of the guiding tool of FIGS. 7 and 8 with the dorsal surface of each of the interbody spacers; and

FIG. 11 is a schematic view showing specific examples of the engagement state between the interbody spacer and the guide rail portion of the guiding tool of the system according to the present invention.

### DETAILED DESCRIPTION

The embodiments for carrying out the present invention will now be explained with reference to the drawings. In the following explanations, the terms “superior” and “inferior” denote an upper side and a lower side in the drawings, respectively. The terms “superior and inferior” are used for the sake of convenience, and upon placement, the superior and inferior sides may be reversed, or the positioning may be horizontal.

FIG. 2 is a schematic view of the state in which a guide rail portion 31 of a guiding tool 30 is inserted and placed in the intervertebral space between a cranial vertebral body 1A and a caudal vertebral body 1B, which is viewed from the ventral side. The operator grips the gripping portion 32 and performs insertion. Actually, the intervertebral space between the cranial vertebral body 1A and the caudal vertebral body 1B is surrounded by tissue called an annulus fibrosus, and thus cannot be seen from the ventral side. However, in this view, the annulus fibrosus is omitted so that the state of placement can be recognized.

FIG. 3 is a perspective view of an interbody spacer according to the present invention as viewed from the ventral side. A superior surface 13 is in contact with the caudal surface of the cranial vertebral body 1A (FIG. 2), while an inferior surface 14 is in contact with the cranial surface of the caudal vertebral body 1B. A lateral surface 11 and a medial surface 12 are located at both end portions along the central axis CL. The overall shape is such that the ventral side is convexly curved. A side surface on the ventral side is a ventral surface 17, and a concave side surface is a dorsal surface 18. The superior surface 13 and the inferior surface 14 are carved with grooves or the like for having the superior surface 13 and the inferior surface 14 firmly joined to the cranial vertebral body 1A and caudal vertebral body 1B, respectively, but these grooves or the like are omitted in FIG. 3.

The interbody spacer 10 has two holes 16 penetrating the superior surface 13 and the inferior surface 14, and these two holes are partitioned by a middle section 15. These holes are called “bone-graft sites,” and are to be filled with a highly osteophilic material such as patient's autogenous bone or artificial bone. An engagement portion 20, to be engaged with a guiding tool to be described later, is formed in the ventral surface 17 in FIG. 3. The number of these holes is not limited to two. Moreover, these holes are not essential in the present invention, and the interbody spacer may be one without any hole.

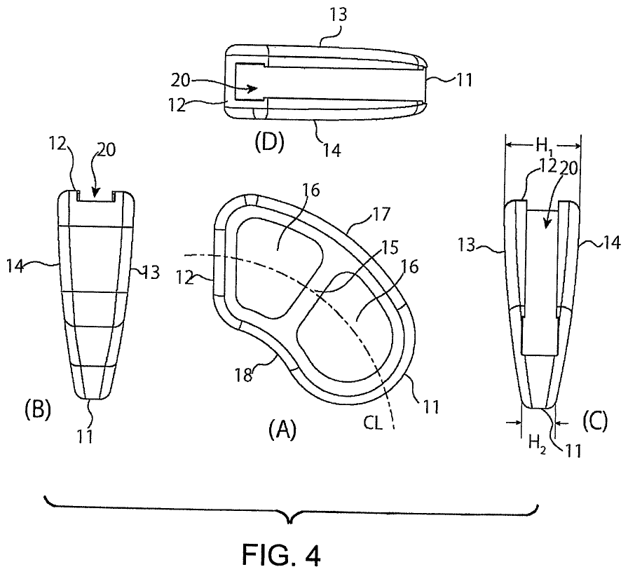

FIG. 4 is a plan view (A), a medial side view (B), a lateral side view (C), and a ventral side view (D) of the interbody spacer 10 In the lateral side view, (C), H₁ is the height of the ventral surface 17, and H₂ is the height of the dorsal surface 18.

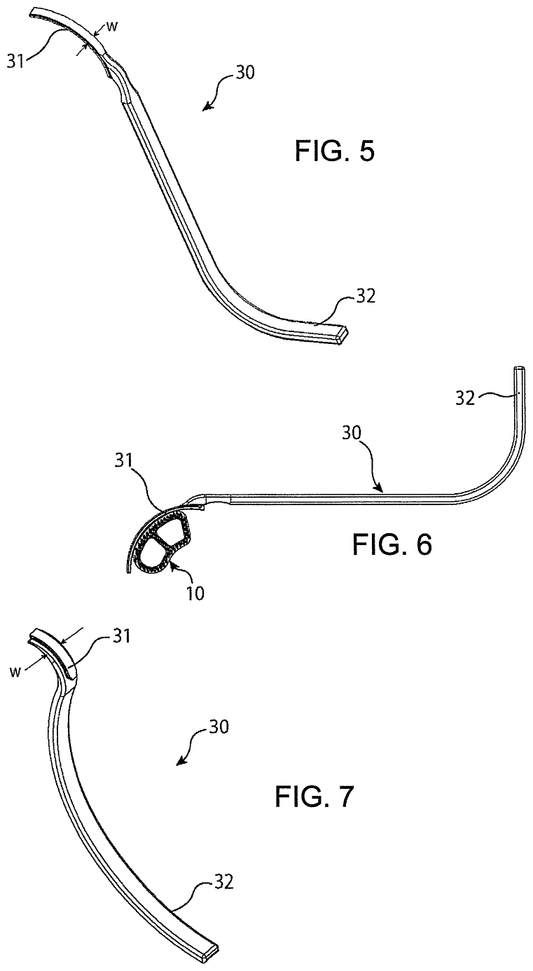

FIG. 5 shows, in perspective view, a first version of the guiding tool 30, and FIG. 6 shows the guiding tool in plan view. A guide rail portion 31 to be engaged with the engagement portion 20 of the interbody spacer 10, and a gripping portion 32 to be gripped by the operator, are formed in the guiding tool 30. FIG. 6 shows a state in which the engagement portion 20 formed in the ventral surface 17 of the interbody spacer 10 is engaged with the guide rail portion 31. W in FIG. 5 denotes the transverse width of the guide rail portion 31.

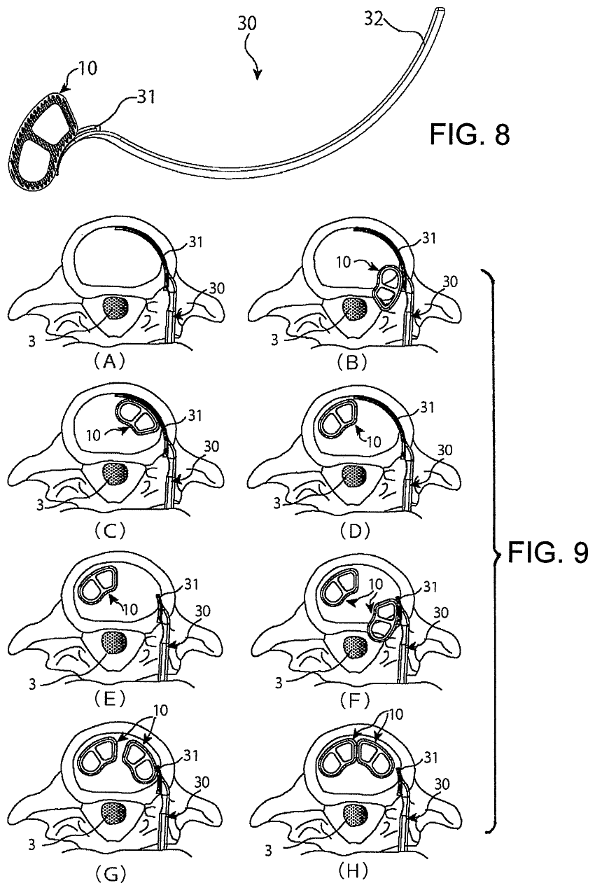

FIG. 7 is a perspective view of a second version of the guiding tool 30, and FIG. 8 is a plan view of the guiding tool 30 of FIG. 7. The guide rail portion 31 to be engaged with the engagement portion 20 of the interbody spacer 10, and the gripping portion 32 to be gripped by the operator, are formed in the guiding tool 30. FIG. 8 shows a state in which the engagement portion 20 formed in the dorsal surface 18 of the interbody spacer 10 is engaged with the guide rail portion 31. W in FIG. 7 denotes the transverse width of the guide rail portion 31.

FIG. 9 schematically shows the steps of placing two interbody spacers 10 between vertebral bodies using the guiding tool 30 of FIGS. 5 and 6. In FIG. 9(A), the guiding tool 30 is placed in a predetermined position while avoiding nerves 3. In FIG. 9(B), the engagement portion 20 of the ventral surface (surface 17 in FIG. 3 and view A in FIG. 4) on the lateral surface 11 side of the interbody spacer 10 is engaged with the guide rail portion 31. In FIG. 9(C), a pusher (not shown) is used to push the medial surface 12 side to insert the interbody spacer 10 along the guide rail portion 31, and in FIG. 9(D), the first one of the interbody spacers 10 is placed in a predetermined position.

Thereafter, the guiding tool 30 is pulled out, and, in FIG. 9(E), a new guiding tool 30 with a shorter guide rail portion 31 is set in a predetermined position between the vertebral bodies. In FIG. 9(F), the engagement portion 20 of the ventral surface 17 on the medial surface 12 side of the interbody spacer 10 is engaged with the guide rail portion 31. In FIG. 9(G), a pusher (not shown) is used to push the lateral surface 11 side to insert the interbody spacer 10 along the guide rail portion 31, and in FIG. 9(H), the second one of the interbody spacers 10 is placed in a predetermined position, and then the guiding tool 30 is pulled out.

The guide rail portion 31 of the guide of FIGS. 5 and 6 is placed against the annulus fibrosus of the intervertebral disc on the ventral side of intervertebral space so as to serve as a wall on the ventral side, so that it is possible to prevent the interbody spacer from perforating the annulus fibrosus and being dislocated to the ventral side from the intervertebral space.

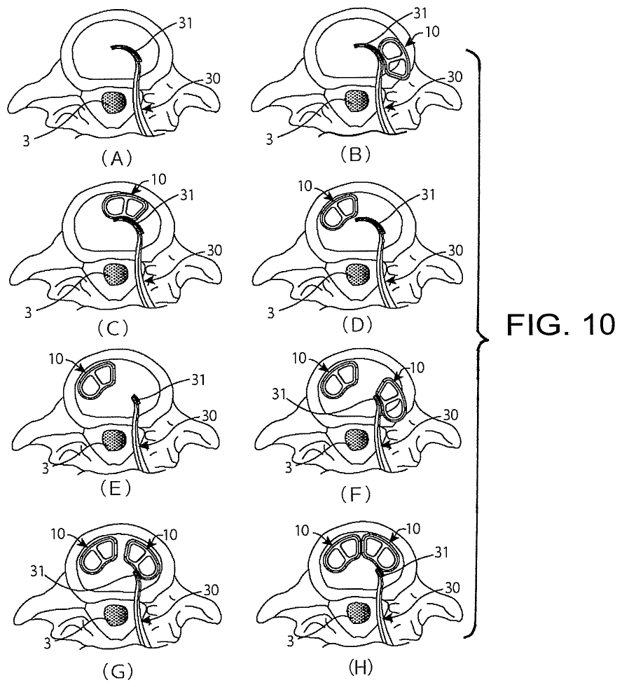

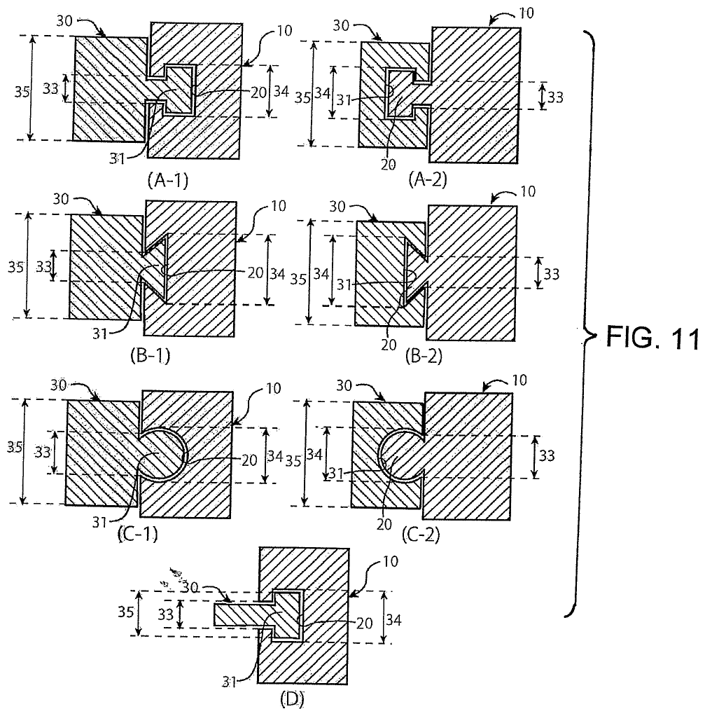

FIG. 10 schematically shows the steps of placing two interbody spacers 10 between vertebral bodies using the guiding tool 30 of FIGS. 7 and 8. This guiding tool is configured to engage with the engagement portion 20 formed in the dorsal surface 18 of the interbody spacer 10, which differs from the guiding tool 30 of FIGS. 5 and 6. With this configuration, the nerves 3 are protected by the guiding tool 30.

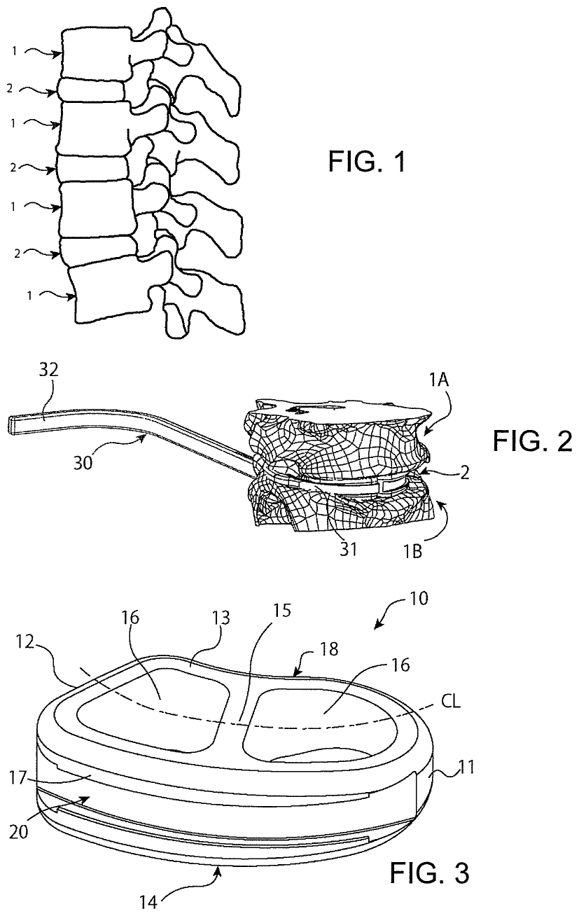

FIG. 11 schematically shows specific examples of the engagement state between the interbody spacer 10 and the guide rail portion 31 of the guiding tool 30. In each of these examples, reference number 33 designates the width, in transverse cross-section, of the opening of a concave groove that serves either as a guide rail portion 31 or as an engagement portion 20. Reference number 34 designates the maximum width of the same concave groove. Reference number 35 designates the transverse width of the guiding tool. In FIG. 11(A1), the cross-section of the guide rail portion 31 is a T-shaped convex cross-section, and the cross-section of the opposing engagement portion 20 of the interbody spacer 10 is a T-shaped concave cross-section. FIG. 11(A-2) shows the reverse of FIG. 11(A-1).

FIG. 11(B-1) and FIG. 11(B-2) show an engagement structure of a dovetail groove, and FIG. 11(C-1) and FIG. 11(C-2) show an engagement structure having a ball-and-socket relation. These engagements have their own characteristics, and the T-shape and the dovetail groove shape are characterized in having an advantage in the processing cost and capability of secure retention, while the ball-and-socket shape has a better sliding property.

FIG. 11(D) is a modification of FIG. 11(A-1), in which the transverse width of the guide rail portion 31 is extremely small compared to the height of the ventral surface and/or the dorsal surface of the interbody spacer, and the interbody spacer 10 can be more easily and accurately placed in a predetermined position even in a narrow area between vertebral bodies.

### INDUSTRIAL APPLICABILITY

The system according to the present invention is a simple system, and using this system makes it easy to place an interbody spacer accurately in a predetermined position between vertebral bodies even in a narrow surgical field.

### DESCRIPTIONS OF REFERENCE NUMERALS

1: vertebral body
1A: cranial vertebral body
1B: caudal vertebral body
2: intervertebral disc
3: nerves
10: interbody spacer
11: lateral surface
12: medial surface
13: superior surface
14: inferior surface
15: middle section
16: bone-graft site
17: ventral surface
18: dorsal surface
20: engagement portion
30: guiding tool
31: guide rail portion
32: gripping portion
33: opening width
34: maximum width
35: transverse width

1. A system for treating a spinal disease, said system comprising:
an interbody spacer which is curved in a horizontal direction and used by being inserted between vertebral bodies, the interbody spacer including a pair of contact surfaces to be in contact respectively with each of the vertebral bodies, and including a ventral surface which is a side surface on a ventral side having a convex shape in plan view and which connects said pair of contact surfaces on the ventral side, and a dorsal surface which is a side surface on a dorsal side having a concave shape in plan view and which connects said pair of contact surfaces on the dorsal side, the interbody spacer including an engagement portion, which is located along the direction in which the interbody spacer is curved, in one of said ventral and dorsal surfaces; and
a guiding tool for guiding the interbody spacer to a predetermined final position between the vertebral bodies, the guiding tool having proximal and distal portions and including, on said distal portion, a guide rail portion to be fitted to said engagement portion, said guide rail portion having a radius of curvature in plan view which is the same as a radius of curvature of said one of said ventral and dorsal surfaces in plan view, and a gripping portion at said proximal portion, the gripping portion being capable of being gripped by an operator at a location external to the patient to hold said guide rail portion in a fixed position between said vertebral bodies while said interbody spacer is being inserted between said vertebral bodies and being guided by said guide rail portion;
wherein one of the transverse cross-section of the engagement portion and the transverse cross-section of the guide rail portion has a concave shape with an opening, while the other has a convex shape that corresponds to the concave shape of said one of said transverse cross-sections, and the concave shape of said one of said transverse cross-sections includes a portion having a maximum width that is greater than the width of the opening of said concave shape of said one of said transverse cross-sections; and
wherein said distal portion of the guiding tool is removable from said interbody spacer, by said operator's pulling on said gripping portion, while said interbody spacer remains in said predetermined final position.

2. The system according to claim 1, wherein the guide rail portion has a maximum transverse width equal to or smaller than the height of the one of said ventral and dorsal surfaces having said engagement portion.

3. The system according to claim 1, characterized in that the transverse cross-section of the engagement portion is a T-shaped concave cross-section, and the transverse cross-section of the guide rail portion is a T-shaped convex cross section.

4. The system according to claim 2, characterized in that the transverse cross-section of the engagement portion is a T-shaped concave cross section, and the transverse cross-section of the guide rail portion is a T-shaped convex cross-section.

5. The system according to claim 1, characterized in that the transverse cross-section of the engagement portion is a T-shaped convex cross-section, and the transverse cross-section of the guide rail portion is a T-shaped concave cross-section.

6. The system according to claim 2, characterized in that the transverse cross-section of the engagement portion is a T-shaped convex cross-section, and the transverse cross-section of the guide rail portion is a T-shaped concave cross-section.

7. A method for insertion of an interbody spacer between vertebral bodies for treatment of a spinal disease, said method comprising:
inserting and guiding an interbody spacer into a final predetermined position between two vertebral bodies, the interbody spacer being curved in a horizontal direction and including a pair of contact surfaces to be in contact respectively with each of said vertebral bodies, and including a ventral surface which is a side surface on a ventral side having a convex shape in plan view and which connects said pair of contact surfaces on the ventral side, and a dorsal surface which is a side surface on a dorsal side having a concave shape in plan view and which connects said pair of contact surfaces on the dorsal side, the interbody spacer including an engagement portion, which is located along the direction in which the interbody spacer is curved, in one of said ventral and dorsal surfaces;
wherein said guiding of the interbody spacer to said predetermined final position between said vertebral bodies is carried out by means of a guiding tool having proximal and distal portions and including, on said distal portion, a guide rail portion to be fitted to said engagement portion of said interbody spacer, said guide rail portion having a radius of curvature in plan view which is the same as a radius of curvature of said one of said ventral and dorsal surfaces in plan view, and a gripping portion at said proximal portion, the gripping portion being capable of being gripped by an operator at a location external to the patient to hold said guide rail portion in a fixed position between said vertebral bodies while said interbody spacer is being inserted between said vertebral bodies and being guided by said guide rail portion;
wherein one of the transverse cross-section of the engagement portion and the transverse cross-section of the guide rail portion has a concave shape with an opening, while the other has a convex shape that corresponds to the concave shape of said one of said transverse cross-sections, and the concave shape of said one of said transverse cross-sections includes a portion having a maximum width that is greater than the width of the opening of said concave shape of said one of said transverse cross-sections; and
wherein said distal portion of the guiding tool is removed from said interbody spacer, by pulling on said gripping portion, while said interbody spacer remains in said predetermined final position.

8. The method according to claim 7, wherein the guide rail portion has a maximum transverse width equal to or smaller than the height of the one of said ventral and dorsal surfaces having said engagement portion.

9. The method according to claim 7, characterized in that the transverse cross-section of the engagement portion is a T-shaped concave cross-section, and the transverse cross-section of the guide rail portion is a T-shaped convex cross section.

10. The method according to claim 8, characterized in that the transverse cross-section of the engagement portion is a T-shaped concave cross section, and the transverse cross-section of the guide rail portion is a T-shaped convex cross-section.

11. The method according to claim 7, characterized in that the transverse cross-section of the engagement portion is a T-shaped convex cross-section, and the transverse cross-section of the guide rail portion is a T-shaped concave cross-section.

12. The method according to claim 8, characterized in that the transverse cross-section of the engagement portion is a T-shaped convex cross-section, and the transverse cross-section of the guide rail portion is a T-shaped concave cross-section.
